(19) United States
(12) Patent Application Publication
(10) Pub. No.: US 2007/0155769 A1
(43) Pub. Date: Jul. 5, 2007

(54) Use of 14,15-dihydro-20,21-dinoreburnamenin-14-ol for the treatment and/or prevention of serious depression and sleep/waking cycle disorders

(75) Inventors: Jean-Francois Pujol, Paris (FR); Dinah Weissmann, Paris (FR)

Correspondence Address: FOLEY AND LARDNER LLP;SUITE 500, 3000 K STREET NW, WASHINGTON, DC, 20007, US

(73) Assignee: BIOCORTECH

(21) Appl. No.: 10/587,270

(22) PCT Filed: Jan. 27, 2005
(86) PCT No.: PCT/FR05/00178

§ 371 Date: Jul. 26, 2006

(30) Foreign Application Priority Data
Jan. 30, 2004 (FR) 0400905
Apr. 8, 2004 (FR) 0403694

(51) Int. Cl. A61K 31/4745

(52) U.S. Cl. 514/283

(57) ABSTRACT

The invention relates to a novel therapeutic use of 14,15-dihydro-20,21-dinoreburnamenin-14-ol for the treatment of serious depression in humans, particularly for the treatment of a patients resistant to conventional anti-depressant treatments and for treatment of sleep/waking cycle disorders.

The purpose of the invention is a new therapeutic application of 14,15-dihydro20,21-dinoreburnamenin14-ol for the treatment of major depression in Man, particularly for treatment of a patient resistant to treatment by conventional antidepressants, or for treatment of wakening-sleep cycle disorders.

Depression is one of the most frequent psychological problems. In France, the proportion of depressive people is 14.9%, including one-third of whom are not treated medically. One woman out of five is affected.

The prevalence of declared depression has been multiplied by a factor of 6 since 1970. The proportion of persons suffering from depression increased particularly among the young between 20 and 29 years old (+65%) between 1992 and 1997, who also suffered from an increase of 20% in the unemployment rate during the same period.

The risk of suffering from a major depression during a lifetime varies from 10 to 25% for women, and from 5 to 12% for men, according to different studies.

Major depression is one of the categories of depressions listed in the DSM IV (American classification of mental disorders) and is characterised by the symptoms of the depression. In particular, a major depression, formerly called melancholic depression, should be distinguished from other clinical groups such as reactional depressive conditions, depressions due to exhaustion, depressions related to the field (depression of a child, pregnant woman, elderly person) or seasonal depressions.

Therefore it is particularly essential that treatments better adapted to this type of depression should be found, particularly because some patients do not respond to conventional antidepressants.

Derivatives of 20,21-dinoreburnamenine, including 14,15-dihydro20,21-dinoreburnamenin14-ol, are already known for their vaso-expanding properties, particularly cerebral, and for their activity in regulation of tyrosine hydroxylase in the locus coeruleus (Bourde et al., Neurochem. Int., 23 (6), 567-574, 1993). They are used for cerebral vasculopathies and for all syndromes caused by alteration of cerebral circulation.

These derivatives and their first known therapeutic application were described in patent application FR 2 381 048, published on Sep. 15, 1978. This patent application has been the object of an additive certificate application FR 2 433 528 published on Mar. 14, 1980.

More particularly, application FR 2 381 048 describes derivatives of 20,21-dinoreburnamenine and their preparation process. The pharmacological properties of these compounds are also described: these compounds are valuable cerebral oxygenators and vasoregulators that in particular increase cerebral flow in the cerebral microcirculation.

Application FR 2 433 528 also describes the process for preparation of a particular isomer derived from 20,21-dinoreburnamenine, and the isomer obtained by this process.

Application WO 89/04830, published on Jun. 1, 1989 describes new substitute derivatives of 20,21-dinoreburnamenine, the process for their preparation and their application as a medicine particularly as an antidepressant.

Depression is a pathological psychic condition combining a stressful mood change and slowing of intellectual activity and motricity. It is a morbid condition, more or less long term, characterised by a certain sadness and reduction of the energy tonicity.

The main symptoms used to diagnose depression in a person are:
depressive mood,
marked reduction of interest or pleasure,
problems in feeding,
sleep problems,
agitation or slowed psychomotricity,
tiredness or loss of energy,
lack of self-esteem or an excessive feeling of culpability,
a reduction of the ability to think or concentrate, or uncertainty
morbid thoughts (60% of cases),
suicidal thoughts (in 15% of cases).

Causes of depression include:

1/ The Hereditary Factor

Persons whose close relatives suffer from or have suffered from depression are most likely to be affected. They have a 15% risk of developing depression, while persons whose close relatives are not depressive only have 2 to 3% risk of developing a depression.

2/ The Biochemical Factor

Current research on depression applies to neurotransmitters. It has thus been noticed that a serotonin deficiency or unbalance caused sleep loss and reduced appetite, and also that a reduction in noradrenaline has an effect on loss of energy, loss of pleasure.

3/ Environmental Factors

Children who have experienced the loss of a loved one such as their parents are more likely to develop depression later in their life. Difficulties in relations, communication problems and family, professional or other conflicts may also contribute to solitude, alienation and result in depression. Financial difficulties and other tensions can also have an important impact.

Seasonal factors must not be neglected: the depression rate is higher during months in which sunshine is lowest.

Prior art describes two major types of treatment for depression.

Firstly, treatment by medicine with antidepressants, appropriate for all forms of depression. They act on the equilibrium of neurotransmitters.

Antidepressants are efficient in 75% of persons suffering from severe depression.

And secondly, psychotherapies—that help patients but cannot be used as the sole treatment.

There are other forms of treatment such as behavioural or cognitic therapies (particularly applicable for neurotic depressions), sismotherapy and electroshock (used as last resort).

The development of depression is very variable and depends on many parameters: etiology, personality of the patient, etc.

If no treatment is given, it often arises that a depression can last 6 months or more, occasionally ending in the extreme termination of suicide. Up to 15% of patients with a serious depression disorder commit suicide.

Depression may be diagnosed using the DSM IV criteria (Diagnostic and Statistical Manual of Mental Disorders, 4th edition, American Psychiatric Association Publisher; Washington D.C.); the DSM IV is a diagnostic and statistical baseline for mental disorders, produced by the American Psychiatry Association.

According to the DSM IV criteria, major depression (also called MDD for <<Major Depressive Disorder>>) is different particularly from dysthymic disorders (minor depression) characterised by a chronic depression but less severe than a major depression, for which the episode can last at least two years, and can degrade into a major depression in more than two-thirds of all cases.

According to the DSM IV criteria, severe depression that is the severe and most common form of depression and for which only 10 to 25% of patients search for treatment, is characterised by one or several episodes of mood change or loss of interest for at least two weeks accompanied by at least four additional symptoms of depression; these symptoms may for example be a change in appetite, weight, sleep or psychomotricity activity; reduction of energy, a feeling of reduced self-esteem, or culpability, difficulty in thinking, concentrating, making decisions, or recurrent thoughts of death, or ideation of plans or attempts to commit suicide.

To be able to characterise a major depressive episode, a new symptom must be present that was not present before, or that has worsened compared with the condition of the person during a previous episode. Symptoms must persist throughout most of the day, almost every day, for at least two consecutive weeks. This episode must be accompanied by significant clinical distress or a deterioration of the social and occupational behaviour. In some persons with more benign episodes, behaviour may appear normal but requires a particularly large effort.

By definition, a major depressive episode is not due to the direct physiological effects of drug abuse (for example in a context of withdrawal/dependence following intoxication with alcohol or cocaine) nor to secondary effects when taking medicine or treatments (for example steroids), nor to exposure to a toxin. Similarly, the episode is not due to direct physiological effects of a medical condition in general (for example hyperthyroidism).

Major depressions include depressions that resist to treatment by classical antidepressants (called TRD for <<Treatment Resistant Depression>>). 30 to 46% of patients suffering from depression have a partial response or no response to antidepressants (Fava et al., Psychiatric Clin. North Am., 19, 2, 179-200, 1996).

Classical antidepressants currently and frequently marketed belong to the following main classes:
tricyclic antidepressants (TCA),
monoamine oxidase inhibitors (MAO) (MAOIs),
selective serotonin recapture inhibitors (SSRIs),
serotonin and noradrenaline recapture inhibitors (SNDRIs),
noradrenaline and selective serotonin antidepressants (NASSAs),
and serotonin receptor modulators.

Treatment resistant depressions (TRD) are a more handicapping and chronic form of MDD (Komstein et al., J. Clin. Psychiatry, 62, suppl. 16, 18-25, 2001). According to the model proposed by Thase et al. (Thase et al., J. Clin. Psychiatry, 58, suppl. 13, 23-29, 1997), the stages of the TRD may be evaluated as follows:
Stage I: Failure in at least one appropriate test of a major class antidepressant;
Stage II: Stage I failure plus failure in an appropriate test of an antidepressant in a class different from that used in stage I;
Stage III: Stage II failure plus failure in an appropriate test of a tricyclic antidepressant;
Stage IV: Stage III failure plus failure in an appropriate test of an MAO inhibitor; and
Stage V: Stage IV failure plus failure in treatment by bilateral electroshock therapy (ECT).

The Massachusetts General Hospital (MGH) Boston has determined a method of classifying the TRD process starting from:
Item 1: no response for each test of a commercial antidepressant generating a global resistance score (1 point per test) (at least 6 weeks of an appropriate dose of antidepressant);
Item 2: optimisation of proportioning, optimisation of the duration and increase/combination of each test (based on the MGH or response to the antidepressant treatment questionnaire) (0.5 point per test and per optimisation/strategy); and
Item 3: the ECT increases the total by 3 points.

The fact that the TRD is a relatively common event in clinical practice should be noted, with more than 50 to 60% of patients not making appropriate responses after an antidepressant treatment.

These major depressions include <<Major Recurrent Depressive Disorders>>(MRDD), associated with hypomaniac episodes.

The severity of these depressive disorders, from minor to severe form, may be evaluated using classic and validated numeric scales, such as the HAMD (<<Hamilton Depression Scale>>) scale or the MADRS (Montgomery and Asberg Depression Rating Scale) scale that are the most frequently used. According to these scales, a depression will be considered as being severe if the symptoms result in a score of more than 26 for the HAMD scale or 35 for the MADRS scale.

Sleep disorders affect an increasing proportion of the population. The proportion of the population suffering from sleep disorders in Europe, the United States and Australia is estimated to be at least 20%. Two studies dealing with large samples of the French population find a prevalence ratio of 22%. One out of every six French people complains about sleep disorders (more than 9 million people).

The severity and chronicity of sleep disorganisation increases with age, 60 to 70% of regular consumers of hypnotics and tranquillisers are more than 40 years old. However, the severity of insomnia in children is not well understood, since investigations are usually based on the evaluation of parents who underestimate disorders. A sleep questionnaire addressed to teenagers between 16 and 19 years old showed that 14% of them experience difficulties in getting to sleep, 8% frequently wake up at night and 6% wake up too early in the morning.

Thus, it is important to have compounds capable of treating major depression disorders in a patient or to treat <<TRD>> patients suffering from depression, particularly major depression, who are resistant to treatment using classical antidepressants as mentioned above, and/or to prevent the treatment of disorders in the wake-sleep cycle.

This is the purpose of the invention described and claimed below.

Surprisingly, it has been discovered that 14,15-dihydro20,21-dinoreburnamenin14-ol, referred to as BC19 in this document, when in the form of a racemic mix, can be used to treat patients suffering from major depression and/or TRD and/or to prevent the treatment of disorders in the wake-sleep cycle.

Surprisingly, it has also been demonstrated that the use of 14,15-dihydro20,21-dinoreburnamenin14-ol could be given to patients suffering from major depression who were resistant to conventional antidepressant treatments, to make them sensitive to these treatments.

Therefore, the purpose of this invention is the use of a compound with formula (I)

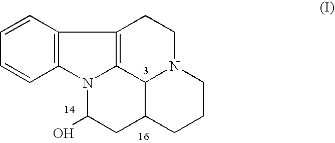

or one of its pharmaceutically acceptable salts for the preparation of a pharmaceutical composition for the treatment or prevention of major depressions (MDD), and/or for the treatment of disorders in the wake-sleep cycle.

Preferably, the said disorders in the wake-sleep cycle are chosen from among narcolepsy, hypersomnia, and chronic hypo-arousal condition.

According to another aspect, the purpose of the invention is the use of a compound with formula (I) or one of its pharmaceutically acceptable salts, for the preparation of a pharmaceutical composition for treatment or prevention for patients suffering from depression and who are partially or totally resistant to treatment by classical antidepressants (patients suffering from TRD), such as antidepressants belonging to the class consisting of tricyclic antidepressants (TCA), monoamine oxidase inhibitors (MAOIs), selective serotonin recapture inhibitors (SSRIs), serotonin and noradrenaline recapture inhibitors (SNDRIs), noradrelanine and selective seretonine antidepressants (NASSAs) or serotonin receptor modulators.

According to one preferred aspect, the invention relates to the use of a compound with formula (I) or one of its pharmaceutically acceptable salts, for preparation of a pharmaceutical composition for the treatment or for prevention for patients suffering from major depression and partially or totally resistant to treatment by conventional antidepressants (patients suffering from MDD and TRD).

According to one particular aspect, the invention relates to the use of a compound with formula (I) or one of its pharmaceutically acceptable salts according to the invention, characterised in that the major depression is a bipolar type depression according to the DSM IV nomenclature, and particularly a major recurrent depressive disorder (MRDD).

According to one particular aspect, the invention relates to the use of a compound with formula (I) or one of its pharmaceutically acceptable salts according to the invention, characterised in that the severity of the depression has a score of more than 26 when it is evaluated using the HAMD (<<Hamilton Depression>>) scale, or more than 35 when evaluated by the MADRS (Montgomery and Asberg Depression Rating Scale) scale.

According to yet another aspect, the purpose of the invention is the use of a compound with formula (I) or one of its pharmaceutically acceptable salts, for preparation of a pharmaceutical composition to treat patients suffering from major depression and resistant to classical antidepressant treatments, to make them sensitive to these treatments.

Pharmaceutically acceptable additive salts include for example additive salts with mineral or organic acids, particularly salts formed with hydrochloric, hydrobromic, hydroiodic, nitric, sulphuric, phosphoric, acetic, formic, benzoic, maleic, fumaric, succinic, tartaric, citric, oxalic, glyoxylic, aspartic, ascorbic acids, alkoylmonosulfonic acids such as methanesulfonic acid, ethane sulfonic acid, propane sulfonic acid, alkoyldisulfonic acids such as methanedisulfonic acid, α,β-ethanedisulfonic acid and arylmonosulfonic acids such as benzenesulfonic acid and aryldisulfonic acids, these salts being mentioned for illustrative purposes only and not forming a limitation.

The compound with formula (I) is characterised by two enantiomeric forms 3α and 16α, and for each of these enantiomeres is characterised by a pair of diastereoisomers according to the configuration of carbon 14:
the (3α,14α)14,15-dihydro20,21-dinoreburnamenin14-ol and (3α,14β)14,15-dihydro20,21-dinoreburnamenin14-ol pair; and
the (14α,16α)14,15-dihydro20,21-dinoreburnamenin14-ol and (−)(14β,16α)14,15-dihydro20,21-dinoreburnamenin14-ol pair.

Rotation capacities have been measured, to determine (+) and (−) signs assigned to each isomer.

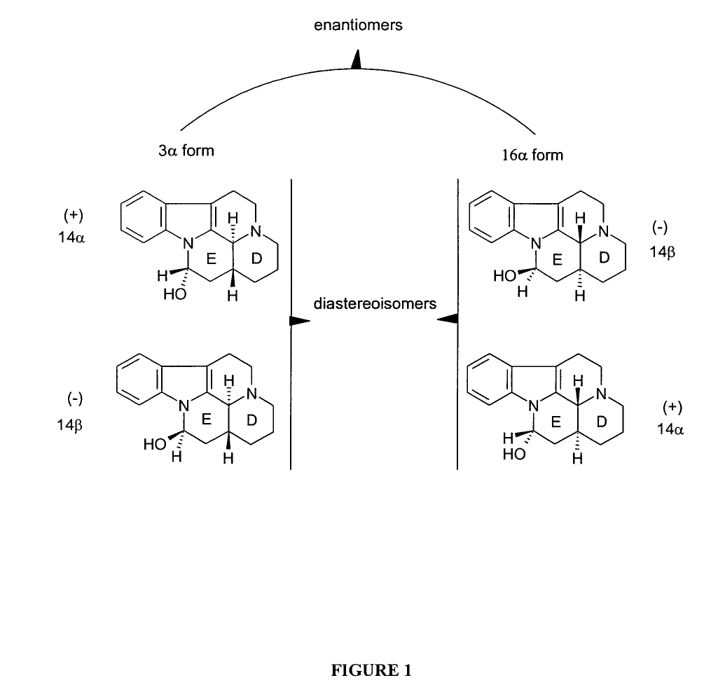

The different compounds are described in FIG. 1 in the article published by Bourde et al. (Neurochem Int., 1993, 23, 567-574).

Therefore, the purpose of one particular aspect of the invention is a compound with formula (I) or one of its pharmaceutically acceptable salts, in which the compound with formula (I) or one of its pharmaceutically acceptable salts is in the form of a racemic or optically active mix.

Another purpose of the invention is use according to this invention, characterised in that the compound with formula (I) or one if its pharmaceutically acceptable salts is chosen from among the following compounds with formula (I):

a) (3α)(±)14,15-dihydro20,21-dinoreburnamenin14-ol; and

b) (16α)(±)14,15-dihydro20,21-dinoreburnamenin14-ol, and in which the mix of the two (+) and (−) diastereoisomers present in these compounds a) and b) is or is not in equimolar proportion.

Another purpose of the invention is the use according to this invention, characterised in that the compound with the formula (I) or one of its pharmaceutically acceptable salts is chosen from among the following compounds with formula (I):

a) (3α,14α)14,15-dihydro20,21-dinoreburnamenin14-ol;

b) (3α,14β)14,15-dihydro20,21-dinoreburnamenin14-ol;

c) (14α,16α)14,15-dihydro20,21-dinoreburnamenin14-ol; and

d) (14β,16α)14,15-dihydro20,21-dinoreburnamenin14-ol.

More particularly, the purpose of the invention is the use of a compound with formula (I) or one of its pharmaceutically acceptable salts according to this invention, for the preparation of a pharmaceutical compositions that can be administrated orally, intravenously, or by an intraperitoneal or intramuscular method, or by any other method for obtaining an antidepressive effect according to this invention, or making patients suffering from major depression who were resistant to classical antidepressant treatments, sensitive to these treatments.

Active substances of pharmaceutical compositions according to the invention may be in any of the oral galenical forms normally used including tablets, capsules and liquid preparations such as elixirs and suspensions containing various colour, taste and stabilisation masking substances.

To produce oral galenical forms according to the invention, the active substance may be mixed with various conventional materials such as starch, calcium carbonate, lactose, sucrose and dibasic calcium phosphate to facilitate the encapsulation process. Magnesium stearate as an additive, provides a useful lubrication function if necessary.

Active substances of pharmaceutical compositions according to the invention may be dissolved or present in suspension in a pharmaceutically acceptable sterile liquid such as sterile water, a sterile organic solvent or a mix of these two liquids. Preferably, such a liquid is appropriate for parenteral injection.

When the active substance is sufficiently soluble, it can be dissolved in a normal saline solution such as a pharmaceutically acceptable sterile liquid; if it is not sufficiently soluble, it can be dissolved in aqueous solutions of an appropriate organic solvent, for example propylene glycol or polyethylene glycol. Aqueous propylene glycol containing 10 to 75% by weight of glycol is usually appropriate. In other examples, other compositions can be obtained by dispersing the active substance as a very fine concentrate in an aqueous carboxymethylic solution of starch cellulose or sodium, or in an appropriate oil, for example peanut oil.

Liquid pharmaceutical compositions such as sterile solutions or suspensions can be used for intramuscular, intraperitonal or subcutaneous injections.

Preferably, the pharmaceutical composition is in the form of unit doses, for example such as tablets or capsules. In this form, the composition is subdivided into unit doses containing appropriate quantities of active substance; unit doses may be packaged compositions, for example powders, flasks or phials. The quantity of active substance in a unit dose of the composition may be modified or adjusted by 2 mg or less, or by 50 mg or more, depending on the particular need and the activity of the active substance.

The recommended oral dose of 14,15-dihydro20,21-dinoreburnamenin14-ol for man may be 20 to 60 mg/day and this dose may be administered in two or three separate doses, preferably during a meal. Most resistant melancholic patients respond to a dose of 20 mg/day, but 40 mg or even 60 mg may be necessary.

Those skilled in the art also know that methods of administrating compounds according to this invention can change significantly. Apart from other oral administrations, slow release compositions may be preferred. Other administration methods may include but are not limited to intravenous injections, intramuscular and intraperitoneal injections, subcutaneous implants, and mouth, sublingual, transdermal, topic, rectal and intranasal administration.

According to one particular embodiment, the purpose of the invention is the use of a compound with formula (I) or its pharmaceutically acceptable salts according to the invention, characterised in that the daily dose is of 20 to 60 mg in the adult.

A specialist will be able to determine the appropriate dose for each patient; this dose may vary as a function of the age, weight and response to treatment of a given patient. The dose examples given above are representative of the average. However, doses smaller or larger than this average may be administered.

Preparation Process for Compounds with Formula (I)

According to the invention, compounds like those defined in formula (I) may be prepared using the following processes starting from the treatment of optically active compounds with formula (II) 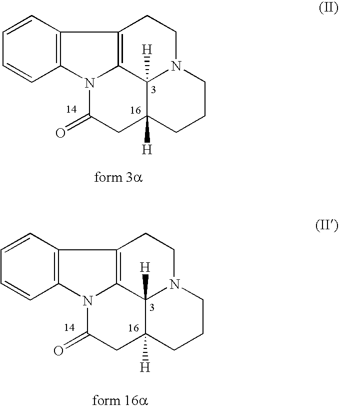
by a reduction agent; the result is two diastereoisomer pairs [(3α,14α),(3α,14β)] and [(^{14α,16}α),(14β,16α)] with formula (I), or a mix of them, and if required, the reaction product is treated by a mineral or organic acid to form the salt.

Products with formula (II) and (II′) may for example be prepared as described in French patent application number FR 2 190 113.

The racemic mix of compounds with formula (II) may be separated by splitting.

A pair of diastereoisomers (±) with formula (I) or mixes of the two diastereoisomers with very variable proportions, may be obtained by reducing one of the two enantiomers with formula (II). The experiment described in French patent application number FR 2 623 503 shows that in practice, only one of the two diastereoisomers is obtained (see example B).

The formula (II) compounds used may be in racemic form or optically active form.

The reduction compound(s) with formula (I) obtained from the product with formula (II) are obviously in the corresponding stereochemical form.

Compounds with formula (II) may be used in the form of one of their additive salts with mineral or organic acids. If this is the case, products with formula (I) may be obtained in salified or non-salified form depending on the chosen operating conditions.

Racemic or optically active mixes of compounds with the general formula (I) may also be prepared as described in French patent application published as number FR 2 381 048 and in the French additive certificate application published as number FR 2 433 528.

Under preferred conditions of the embodiment of the invention, the process described above is carried out as follows.

The reduction agent used may be a hydride, particularly a mixed hydride, for example such as a mixed hydride of lithium and aluminium, sodium and aluminium diethylhydride, sodium hydroboride, lithium hydroboride, diisobutyl-aluminium hydride.

The reduction reaction is carried out using an organic solvent or a mix of solvents, for example such as an ether like ethylic ether, tetrahydrofuran, or an aromatic hydrocarbon such as toluene, benzene, xylene.

The reduction reaction may be carried out at a temperature varying from −20° C. to the reflux temperature of the reaction medium. It is advantageously carried out at ambient temperature.

If used as a reduction agent of a metal hydride, the compound with formula (I) is released from the intermediate complex formed with the hydride using current practice by the addition of an alkaline aqueous solution, for example such as a sodium hydroxide solution.

Reduction of the trans 3α compound (II) may lead to the (+)(3α,14α)14,15-dihydro20,21-dinoreburnamenin14-ol compound.

Reduction of the trans 16α compound (II′) may lead to the (−)(14β,16α)14,15-dihydro20,21-dinoreburnamenin14-ol compound.

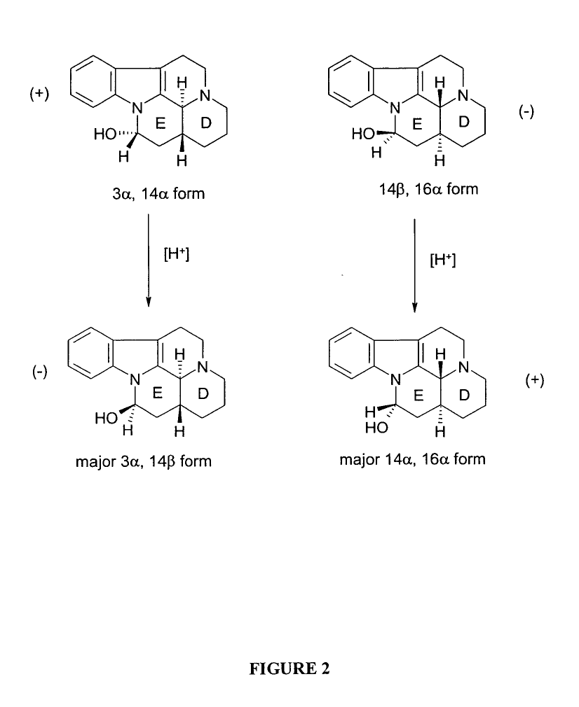

These compounds can be treated by an acid, for example hydrochloric acid, to obtain the most common (−)(3α,14β)14,15-dihydro20,21-dinoreburnamenin14-ol and (+)(14α,16α)14,15-dihydro20,21-dinoreburnamenin14-ol forms respectively (see diagram below and FIG. 2).
Diagram Representing the General Method for Synthesizing Optically Active Isomers of Compounds with Formula (I) from Compounds with Formula (II)(Compounds with Formula (II) Described in the Belgian Patent Application Published as No. BE 764166)

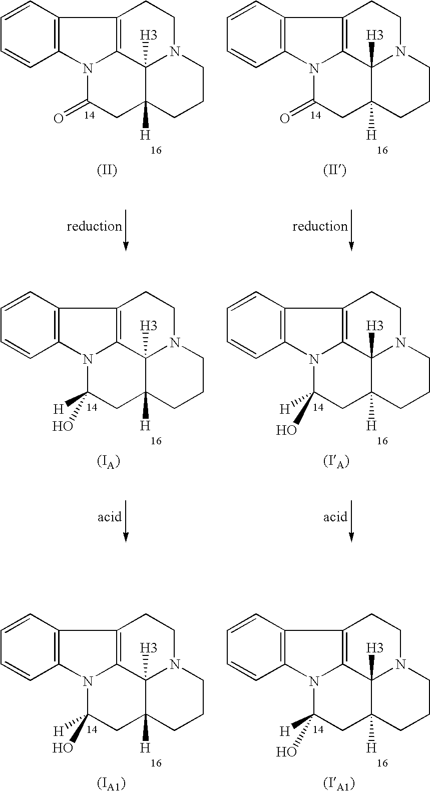

One of the disatereoisomers, or a mix of the disatereoisomers, may be isolated by the usual methods: chromatography, direct crystallisation, differential solubilisation for example such as differential solubilisation in hot toluene.

The legends for the figures and the following examples illustrate the invention without limiting its scope in any way.

Legends for Figures

FIG. 1: Representation of the four forms present in the racemic mix BC19 in acid solution

As indicated, these four forms correspond to two pairs of diastereoisomers that are either in the 16α configuration of the 3α configuration. There is no possible spontaneous transformation from configuration 3α (left) towards configuration 16α (right).

FIG. 2: Representation of the reaction to obtain diastereoisomer (3α,14β) from (3α,14α) and diastereoisomer (14α,16α) from (14β,16β) under the action of an acid.

### EXAMPLES

### Example 1

### Processes for Preparation of Compounds According to the Invention

### Example A

### Processes for Preparation of Racemic Mixes Such as BC19 or Optically Active Mixes According to the Invention

Racemic or optically active mixes of compounds with general formula (I) may in particular be prepared as described in the French patent application number FR 2 381 048 or in the French additive certificate application published as number FR 2 433 528.

### Example B

### Processes for Preparation of Diastereoisomers According to the Invention

The (3α,14α), (3α,14β), (14α,16α) and (14β,16α) diastereoisomers with formula (I) according to the invention may be obtained as described in French patent application number FR 2 623 503. These processes are described briefly below.

### Example B1

### (14β,16α)14,15-dihydro20,21-dinoreburnamenin-14-ol (I′_{A})

10.8 g of (16α)(+)20,21-dinoreburnamenin-14(15H) is dissolved in 110 ml of anhydrous toluene, 18.9 ml of 25% aluminium-sodium diethyl dihydride is added for ten minutes under an inert atmosphere in toluene and this solution is stirred for one hour at ambient temperature. Hydrolysis done by adding 20 ml of 5N soda and heating to 90° C. for two hours. The toluene is distilled and 100 ml water is added simultaneously. The temperature is brought to ambient temperature, the product obtained is spin dried, washed with water, dried at low pressure and 10.7 g of the expected product is recovered that recrystallises in methanol and melts at 254° C. [alpha]_{D}=−36 °±1° (c=0.6% DMF).

Circular dichroism (dioxan): Max. 225 nm Δε=−8 Max. 237 nm Δε=+9,5 Max. 280 nm Δε=−2 NMR spectrum ¹H (pyridine) 250 MHz δ (ppm): 5.78 (H carried by C₁₄). Possible structure with equatorial OH, axial OH not detected.

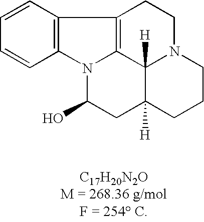

### Example B2

### (3α,14α)14,15-dihydro20,21-dinoreburnamenin-14-ol (I_{A})

The procedure is the same as in example 1 starting from 15 g of (3α)(−)(20,21-dinoreburnamenin-14(15H)-one and 15 g of the expected product is obtained containing very little of the product with axial OH. The product melting at 254° C. is obtained after recrystallisation in methanol.

[alpha]_{D}=+32.5°±1° (c=1% DMF) Circular dichroism (dioxan): Max. 227 nm Δε=+10 Max. 238 nm Δε=−10 Max. 288 nm Δε=+2 NMR spectrum ¹H (pyridine) 250 MHz δ (ppm): 5.79 (H carried by C₁₄). Possible structure with equatorial OH, axial OH not detected.

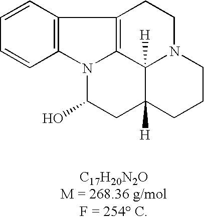

### Example B3

### (14α,16α)14,15-dihydro20,21-dinoreburnamenin-1 4-ol (I′_{A})

2.75 g of the product obtained in example 1 is put into suspension in 55 ml of 2N hydrochloric acid and is heated to 50° C. for one hour and thirty minutes. 55 ml of chilled water is added to the solution obtained and an alkaline pH is obtained by the addition of 10 ml of 22 Be of ammonia and the solution is stirred for 15 minutes at ambient temperature. The precipitate is spin dried, washed with water, dried at 50° C. under low pressure and 2.75 g of product (axial and equatorial OH mix) is obtained. This product is chromatographed under pressure on silica, eluated by an ethyl acetate-methanol-ammonia mix (97-3-0.3). The result obtained is 1.70 g of product (OH axial).

F=234° C. [alpha]_{D}=+150°±2° (c=1% DMF). Circular dichroism (dioxan): Max.: 230 nm Δε=+19 Max.: 290 nm Δε=−1,75 NMR spectrum ¹H (pyridine) 250 MHz δ (ppm): 6.26 (H carried by C₁₄). Possible structure with equatorial OH, axial OH not detected.

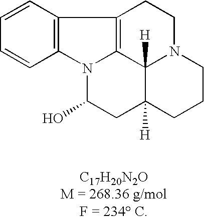

### Example B4

### (3α,14β)14,15-dihydro20,21-dinoreburnamenin-14-ol (I′_{A})

The procedure described in example 3 is followed, starting from 13.3 g of the product obtained in the example 2, and 7.7 g of product is obtained (axial OH).

F 32 234° C. [alpha]_{D}=−152.5°±2.5° (c=1% DMF) Circular dichroism (dioxan): Max.: 228 nm αε=−20 Max.: 290 nm αε=+1.5 NMR spectrum ¹H (pyridine) 250 MHz δ (ppm): 6.23 (H carried by C₁₄). Possible structure with equatorial OH, axial OH not detected.

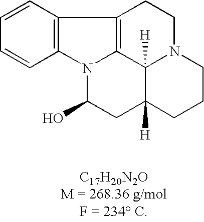

### Example 2

### Pharmaceutical Forms

a) tablets: tablets are prepared using the formula: 14,15-dihydro20,21-dinoreburnamenin14-ol (BC19): 30 mg Excipient q.s. for a tablet (detail of excipient: lactose, wheat starch, treated starch, rice starch, magnesium stearate, talc)

b) capsules: tablets are prepared to the following formula: 14,15-dihydro20,21-dinoreburnamenin14-ol (BC19): 30 mg Excipients: saccharose (115 mg/capsule), starch, stearic acid, lactose, talc, shellac, povidone, methacrylic polymers.

### Example 3

### Pharmacological Study; Determination of Acute Toxicity of BC19 or 14,15-dihydro20,21-dinoreburnamenin14-ol

Acute toxicity is determined on batches of 10 male and female mice, weighing from 20 to 22 g, fasting since the previous evening.

The product is administered intravenously, in solution in physiological saline solution, to which a few drops of hydrochloric acid have been added (products to be tested are then in hydrochloric solution).

Mortality is recorded daily for one week. Values of the lethal doses 50 (LD 50) were determined using the method given by Lichfield J. T. and Wilcoxon F. (J. Pharm. Exp. Therap.96:99, 1949). The results obtained did not demonstrate any toxicity of the BC 19 compound at effective doses.

### Example 4

### Application of BC19 or 14,15-dihydro20,21-dinoreburnamenin14-ol to the Treatment of Wake-sleep Cycle Disorders

The anatomical data presented in table 1 below were obtained three days after a single injection or after a sequential treatment of five injections at a rate of one injection every three days. Immunopositive cells for tyrosine hydroxylase (TH) and fibres containing noradrenaline were identified by immunocytochemistry in the examined brain areas. Sleep records were made on a group of ten Balb/c mice. The electroencephalogram for each animal was recorded continuously for five days for acquisition of basal data about the wake-sleep cycle. Five animals were then treated by five IP injections every three days starting from day 5 until day 17 of the experiment. The other five animals were injected with the excipient at the same time. After the last injection, all mice were deprived of sleep for 6 hours and records went on for another two consecutive days to measure the total REM sleep recovery. The given BC19 dose is 20 mg/kg for each injection, in all these experiments.

TABLE 1
Original parameters modified by the treatment
of consanguine Balb/c mice.

| Parameter | Balb/c controls | Treated Balb/c | Treatment |
|---|---|---|---|
| Total number of | 910 ± 22 | 1228 ± 24 | sacrifice 3 |
| immunopositive | (100 ± 2%) | (135 ± 3%)*** | days after a |
| neurones for | | | single IP |
| TH in LC | | | injection |
| | | | (20 mg/kg) |
| Number of | 552 ± 40 | 671 ± 24 | sacrifice 3 |
| neurones in the | (100 ± 7%) | (121 ± 4%)** | days after a |
| posterior third | | | single IP |
| of the group of | | | injection |
| cells expressing | | | (20 mg/kg) |
| hypocretine in | | | |
| the hypothalamus | | | |
| Density of fibres | 0.032 ± 0.002 | 0.050 ± 0.003 | Sequential |
| containing | (100 ± 7%) | (156 ± 9%) | treatment |
| noradrenaline | | | |
| (μm/μm²) in the | | | |
| prefrontal cortex | | | |

Duration of REM sleep
during the recovery period (mn)

| | | |
|---|---|---|
| Without sleep | 73 ± 10 | |
| deprivation | (100 ± 13%) | |
| (controls) | | |
| During the | 74 ± 9 | 110 ± 8 |
| recovery period | (101 ± 12%) | 151 ± 11*** |
| (deprived mice) | | |

These results demonstrate that when injected into Balb/c mice, the BC19 compound is capable of:
restoring the noradrenergic phenotype in a significant population of locus caeruleus;
restoring noradrenergic innervation in the prefrontal cortex;
restoring the hypocretine phenotype in a sub-population of neurones of the hypothalamus; and
reversing the inability of these consanguine mice to recover REM (<<Rapid Eye Movement>> sleep after sleep privation, REM sleep also being called paradoxal sleep).

Thus, this compound appears as being active in the treatment of disorders in the wake-sleep cycle, particularly including narcolepsy, hypersomnia and a chronic hypo-arousal condition.

1-11. (canceled)

12. A method for treating or preventing a major depression and/or treating a wake-sleep cycle disorder, comprising
administering to a subject in need thereof a pharmaceutical composition comprising a compound with formula (I) or a pharmaceutically acceptable salt thereof: 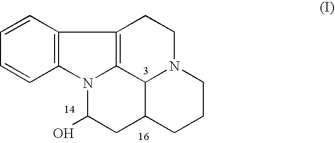
wherein the hydrogen atom in position 3 and the hydrogen atom in position 16 are trans, and the hydroxyl radical in position 14 in an α or β form.

13. The method of claim 12, wherein the subject is partially or totally resistant to classical antidepressants.

14. The method of claim 12, the depression is a bipolar depression according to DSM IV classification.

15. The method of claim 14, wherein the bipolar type depression is a major recurrent depressive disorder (MRDD).

16. The method of claim 12, wherein the subject is suffering from the major depression and is resistant to a classical antidepressant treatment and wherein said administering makes the subject sensitive to the classical antidepressant treatment.

17. The method of claim 12, wherein said wake-sleep cycle disorder is selected from the group consisting of narcolepsy, hypersomnia and a chronic hypo-arousal condition.

18. The method of claim 12, wherein the compound with formula (I) or one of its pharmaceutically acceptable salts is in the form of a racemic or an optically active mix.

19. The method of claim 12, wherein the compound with formula (I) or one of its pharmaceutically acceptable salts is selected from:
a) (3α)(±)14,15-dihydro20,21-dinoreburnamenin14-ol; and
b) (16α)(±)14,15-dihydro20,21-dinoreburnamenin14-ol;
and wherein (+) and (−) diastereoisomers are or are not present in the compound in an equimolar proportion.

20. The method of claim 12, wherein the compound with formula (I) or one of its pharmaceutically acceptable salts is selected from the group consisting of
a) (3α,14α)14,15-dihydro20,21-dinoreburnamenin14-ol;
b) (3α,14β)14,15-dihydro20,21-dinoreburnamenin14-ol;
c) (14α,16α)14,15-dihydro20,21-dinoreburnamenin14-ol; and
d) (14β,16α)14,15-dihydro20,21-dinoreburnamenin14-ol.

21. The method of claim 12, wherein said administering is performed orally, intravenously, or by an intraperitoneal or intramuscular method.

22. The method of claim 12, wherein said administering comprises administering a daily dose from 20 to 60 mg of the compound with formula (I) or a pharmaceutically acceptable salt thereof.
